(19) United States
(12) Patent Application Publication
(10) Pub. No.: US 2008/0194016 A1
(43) Pub. Date: Aug. 14, 2008

(54) Tissue Container, and Devicer and Method For Providing Such a Tissue Container With Data

(76) Inventor: Ronald Johannes Wilhelmus Kusters, Almere (NL)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/576,163

(22) PCT Filed: Oct. 10, 2005
(86) PCT No.: PCT/NL2005/050012

§ 371 Date: Dec. 21, 2007

(51) Int. Cl. C12M 1/16; B01L 3/00; B65D 85/00; B41J 2/435; C12M 3/00

(52) U.S. Cl. 435/307.1; 206/459.5; 422/102; 347/264

(57) ABSTRACT

The invention relates to a container for containing tissue, comprising at least one receiving space for tissue and at least one information surface for arranging data, wherein the information can be arranged by means of a laser. The invention also comprises a device provided with a laser for arranging information on such a container. The invention moreover also comprises a method for arranging information on such a container using a laser.

The invention relates to a container for containing tissue, comprising at least one receiving space for tissue and at least one information surface for arranging data. The invention also comprises a device for arranging information on such a container. The invention moreover comprises a method for arranging information on such a container.

Containers for containing tissue are frequently used to store, order and treat tissue material. Tissue is here understood to mean organic material, isolated from for instance humans, animals or plants. This tissue can for instance be intended for histological or pathological research. Because large quantities of tissue samples are generally examined and analysed in laboratories, it is necessary to be able to trace tissues back to the different sources from which they originate. For this purpose the containers in which the tissue is situated are provided with data from which the origin of the tissue can be traced. This can for instance be a number linked to labels in a log book, a database, or a registration number of a patient. These numbers can for instance be arranged manually by means of a pen. For the sake of legibility it is however recommended to arrange the data using a printer, preferably coupled to an automated database in which the data are stored.

A drawback of arranging data on containers both manually and using a printer is that the arranging is relatively time-consuming, particularly in the case of larger quantities of containers.

In addition to this, both printers and pens or felt-tip pens regularly provide a reduced performance, for instance due to an empty ink reservoir or blocked printer head, whereby the data arranged on the container are difficult to read.

Another drawback of the known methods is that data written or printed with a printer can be erased, for instance due to wear. As a result, the origin of the tissue in the container can no longer be traced. This danger is even greater when the data arranged on the container are exposed to aggressive agents. This is for instance the case when the tissue is pre-treated for microscopic examination. In this sample pre-treatment both the tissue and the container are exposed to diverse chemicals in order to for instance fix and colour the cells in the tissue, whereafter the tissue can be cut into slices for use under a microscope. The container herein comes into contact with organic solvents such as for instance formaldehyde, xylene, acetone, ethyl alcohol, isopropyl alcohol and related substances. Diverse acids and substances such as paraffin and hot wax are also used for specific applications. Under the conditions applied the above stated chemicals can however erase or make illegible the data on the container.

The present invention has for its object to provide a solution for the undesired erasing of data on containers for tissue. The invention also has for its object to make it possible to arrange data more rapidly on such containers.

The invention provides for this purpose a container for containing tissue, comprising at least one receiving space for tissue and at least one information surface for arranging data, characterized in that at least the information surface is manufactured from a material which can be coloured by electromagnetic radiation. By making focussed use of electromagnetic radiation to arrange data on the information surface, the data are arranged non-erasably in the material which can be coloured by radiation. The colouring results from chemical transformation under the influence of the electromagnetic radiation, wherein the electromagnetic radiation can for instance be infrared radiation (radiant heat), light, ultraviolet radiation or any other useable electromagnetic radiation. Suitable materials are commercially available. The radiation is preferably concentrated on a small surface area so that the information can be arranged in a high resolution, and a high information density can thus be achieved. The material is preferably suitable for colouring using laser radiation. Since laser radiation can project very concentrated electromagnetic radiation onto a small surface area, such a material makes it possible to arrange data on the container with a high resolution and thus achieve a high information density. Apart from the choice of material for the information surface, the form of the container can be based on any known usual form of tissue container. These generally take a re-closable form such that in the closed situation a piece of tissue cannot be removed from the receiving space. The receiving space of common containers for tissue is further provided with recesses through which liquids and gases from outside the container can easily come into contact with tissue positioned in the receiving space, wherein the liquids or gases can also leave the receiving space easily. The recesses can be arranged in the form of a grating. Access of liquids and gases is particularly important in sample pre-treatment of a piece of tissue, wherein the marked container is immersed in diverse liquid chemicals, for instance in order to fix the cells of a piece of tissue for microscopic examination. The use of a tissue container according to the invention prevents data arranged on the marked container being erased during the treatment. It will be apparent that to be able to withstand such treatments the tissue container must be manufactured from a suitable chemically-resistant material

It is recommended that the material which can be coloured by electromagnetic radiation is formed substantially from a plastic material. Plastic material can be readily embodied in diverse forms and can be obtained with the desired physical and chemical properties. It is advantageous if the whole container is formed substantially from the plastic material. The entire container can thus be manufactured integrally, which makes the container easy to produce by means of known techniques for the processing plastics, such as injection moulding. In a particular preferred embodiment, the plastic material comprises at least one of the following plastics: acetal copolymer, acrylonitrile butadiene styrene, nylon, polyacetal, polycarbonate, polyester, polypropylene, polyurethane, polystyrene, polyphenylene sulphide, polyethylene terephthalate, polybutylene terephthalate and polyoxymethylene. Such plastics are generally available in readily processable variants. Of the stated plastics diverse variants are available with the desired physical and chemical properties for specific processes. In a very advantageous variant the plastic material substantially comprises polyacetal. A container manufactured from the plastic polyacetal is widely applicable and generally accepted as a safe-to-use material, particularly in the field of histology and pathology. Other names for polyacetal are POM or polyoxymethylene, wherein the polymer substantially comprises (—CH₂O—) as repeating monomer. The two best-known forms of polyacetal are the homopolymer, in which formaldehyde bonds substantially form the chains of the polymer, and the copolymer, in which trioxane, the trimer of formaldehyde, and other monomers are connected. Polyacetal in particular has a good chemical resistance compared to a wide range of common chemicals. This results in an improved durability of data for a wide range of applications arranged on the container by selective colouring of the plastic with electromagnetic radiation. In addition, objects manufactured from polyacetal have good mechanical properties.

In a particular preferred embodiment, the material which can be coloured by electromagnetic radiation comprises a radiation-absorbing pigment. The radiation-absorbing pigment makes it possible to absorb the electromagnetic radiation efficiently, which results in an accelerated colouring of the material which can be coloured by electromagnetic radiation. The radiation-absorbing pigment can for instance be applied to the surface of the information surface. The radiation-absorbing pigment is preferably mixed with the material which can be coloured by the electromagnetic radiation A more homogeneous colouring is thus achieved, whereby data arranged on the information surface are more readily visible. It is recommended that the radiation-absorbing pigment comprises at least one of the following components: mica, pearl pigment, kaolin, aluminium metal, aluminium silicate, antimony trioxide, iron oxide, tin oxide, titanium oxide and aluminium hydroxide. The stated pigments are tried and tested pigments which enable an efficient colouring.

In a particular preferred embodiment, the material which can be coloured by radiation is formed by a laminate comprising at least two layers having mutually contrasting colours. A greater colour variation in the presentation of the data on the container is thus made possible. The two contrasting colours of the layers of the laminate can be chosen freely, wherein the upper layer is selectively removed by electromagnetic radiation and thus reveals the underlying layer in the colour contrasting with the upper layer.

It is advantageous if the information surface can be coupled releasably to the container. Arranging the data on the information surface can thus take place independently of the rest of the container so that a more flexible manner of working is possible. For a releasably coupling connection of the information surface to the container it is possible to envisage diverse systems, such as snap systems or slide systems, with which rapid and simple coupling and uncoupling can be envisaged. The coupling is preferably provided with a locking. Uncoupling is hereby made more difficult, so that the risk of undesired uncoupling of the information surface from the container is minimized. The locking can for instance comprise a barb-like snap element.

In a particular preferred embodiment the container is provided with a reference. It thus becomes possible for instance to determine the position of the information surface of the container in an automated system using detecting means for the reference, the information surface being placed at a known position relative to the reference. The reference can be physical or visual, for instance a physical recess or an optically recognizable symbol.

The invention also provides such a container in accordance with each of the above described preferred embodiments, characterized in that the information surface is provided with data arranged by selective colouring of the material which can be coloured by radiation. Such coloured or burnt-in information is more durable and better resistant to physical and chemical wear than for instance the known printed data.

In addition, the invention provides a device for arranging information on a container for tissue as according to any of the foregoing claims, comprising marking means for arranging data on an information surface of the container, and positioning means for relative positioning of the information surface and the marking means, characterized in that the marking means comprise an electromagnetic radiation source. The use of an electromagnetic radiation source to arrange data on a tissue container according to the invention makes it possible to carry out the marking more rapidly than is possible using known techniques such as a matrix printer. The electromagnetic radiation source is preferably a laser. A high resolution can be achieved with a laser, so that a relatively large amount of data can be arranged on a relatively small information surface. Depending on the material which can be coloured by electromagnetic radiation that is used, diverse common lasers can be chosen, such as a YAG-laser or a CO₂-laser.

In a particular preferred embodiment the positioning means comprise detection means for determining the position of the information surface of the container. The chance of errors in the arranging of data is hereby minimized. The detection means can take both an optical and mechanical form. In order to determine the position of the information surface, this position can for instance be related to a detected position of a peripheral side of the container. The detecting means are preferably adapted for recognition of the reference of the container, wherein the reference can for instance be a protruding element or a border round the information surface.

It is advantageous if the marking means are connected to a database with data. Data, for instance patient numbers, can thus be arranged rapidly and efficiently on a series of containers.

The invention also provides a method for arranging data on a container for tissue according to the invention, comprising the following processing steps of: positioning an information surface of the container and an electromagnetic radiation source relative to each other, and arranging data on the information surface by selective colouring of at least a part of the information surface by means of electromagnetic radiation. Such a method makes it possible to arrange the data on the container in rapid manner. The data are moreover better resistant to erasure by chemical and mechanical influences.

It is recommended that a laser is used as electromagnetic radiation source. A laser makes it possible to achieve a high information density of data on the container. A laser is furthermore able to arrange the data very rapidly.

In a particular preferred embodiment the data are arranged using a device according to the invention. The method can be carried out more efficiently by using such a device. The radiation source can for instance here be controlled from a database with data, enabling operation to be carried out more rapidly and more reliably.

The invention will now be further elucidated on the basis of several non-limitative embodiments.

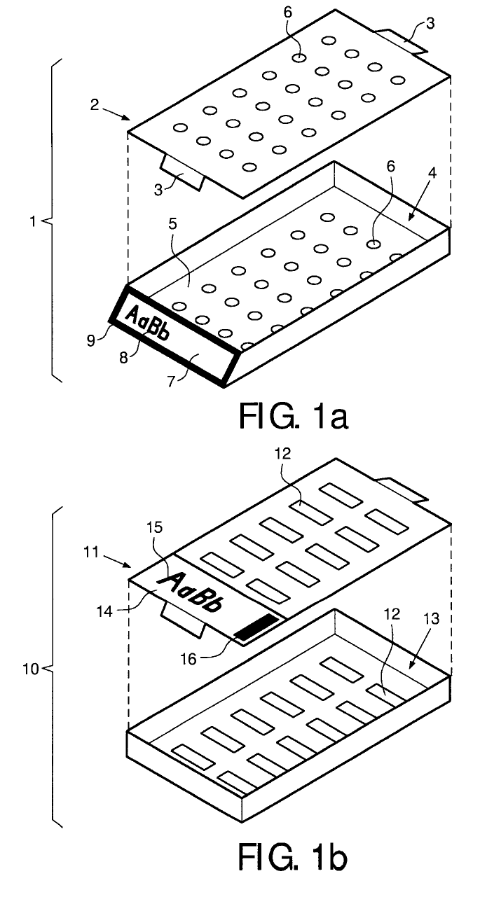

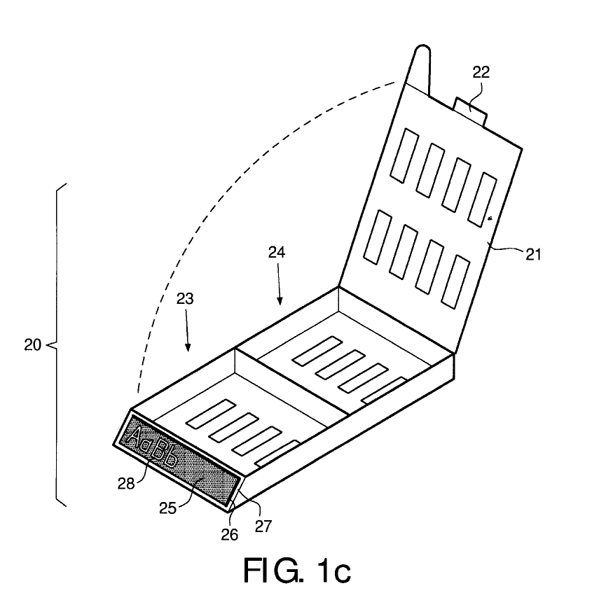

FIGS. 1a-c show diverse containers for tissue according to the invention.

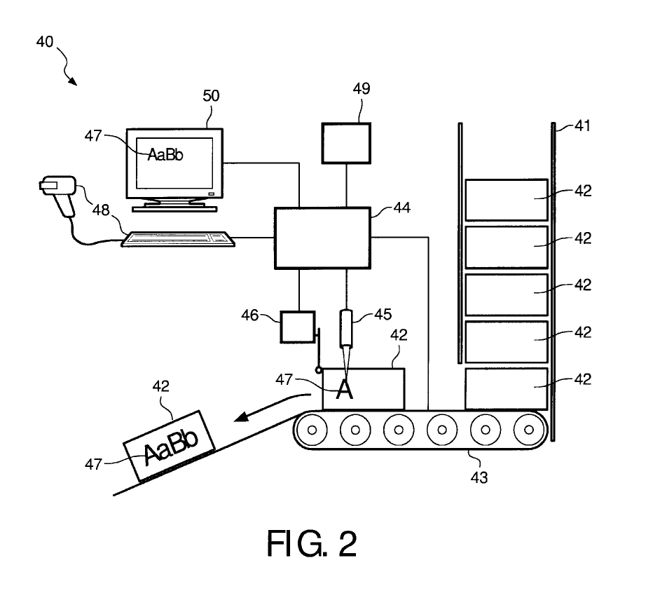

FIG. 2 shows a schematic view of a device for arranging information on containers according to the invention.

FIG. 1a shows a container 1 for tissue according to the invention. Tissue container 1 is provided with a releasable closing element 2 provided with clamping members 3, with which closing element 2 can be snapped fixedly onto a receiving space 4 of container 1 as according to the dotted lines in order to close the container such that tissue (not shown) placed in receiving space 4 is held inside receiving space 4. Such a container 1 can otherwise also be used without closing element 2. Both closing element 2 and base 5 of receiving space 4 are provided with recesses 6, whereby media, in particular liquids and gases, have free access to tissue placed in receiving space 4. Tissue in receiving space 4 can hereby be treated for diverse purposes. In histology and pathology the tissue is for instance treated with diverse liquid chemicals as part of the sample preparation whereby tissue is for instance made suitable for microscopic examination. Since a plurality of tissue samples is usually treated simultaneously during sample preparation, it is important to be able to distinguish the tissue containers. For this purpose tissue container 1 is provided with an information surface 7 on which are arranged data 8 relating to the tissue placed in container 1. In addition to for instance numbers and letters, these data can also comprise alternative representations of data, such as for instance a bar code. It is generally known to arrange such data 8 using ink, applied using for instance a pen or a printer. A drawback is however that data 8 applied using ink, for instance during the sample preparation, can become difficult to read. The container for tissue 1 shown here does not however have this problem, since the entire container is manufactured from a laser-colourable plastic. Data 8 are burnt into the laser-colourable plastic using electromagnetic radiation of a suitable wavelength. The data 8 arranged in this manner are found to be very difficult to erase. The data are even found able to withstand repeated chemical treatments with diverse solvents such as are applied in histology and pathology. The data 8 can be arranged using an automated marking device provided with a laser. To enable automatic arrangement of data 8, the information surface 7 is provided with a dark-coloured reference border 9 which can be recognized by detection means of the automated marking device, so that the laser applies the data 8 to the part of information surface 7 located inside reference border 9.

FIG. 1b) shows a second container for tissue 10, similar to container 1 of FIG. 1a). This tissue container is also provided with a releasable closing element 11, or cover 11, and recesses 12 for admitting liquid and gaseous media to receiving space 13 of tissue container 10. This container 10 is also manufactured wholly from a plastic which can be coloured by electromagnetic radiation. An information surface 14 is placed on releasable cover 11 for the purpose of arranging data 15. To enable localization of the information surface by optical detection equipment, a reference bar 16 is also arranged on information surface 14. The plastic which can be coloured by electromagnetic radiation from which container 10 is manufactured is mixed with a radiation-absorbing pigment, whereby less radiation is necessary to cause sufficient colouring of the plastic, so that arranging of data 15 can take place more quickly. The coloured data 15 moreover have an improved colour contrast with the parts of information surface 14 not exposed to radiation.

FIG. 1c) shows a third container 20 for tissue according to the invention, similar to the tissue containers of FIGS. 1a) and 1b). Closing cover 21 is connected hingedly to the rest of container 20 and is provided with a flexible closing means 22. The container is provided with two separate receiving spaces 23, 24 for tissue, which can both be closed off by the closing cover 21 so that tissue is held inside receiving spaces 23, 24. In contrast to the containers shown in FIGS. 1a and 1b, in this container 20 only the information surface 25 is provided with a material which can be coloured by electromagnetic radiation. The surface of information surface 25 consists of a laminate of a dark-coloured thin layer 26 which is arranged on the underlying light-coloured plastic 27. For the sake of clarity the dark-coloured layer is here shown relatively too thick. By selectively burning away the dark layer 26 with a laser beam, the underlying light-coloured material 27 becomes visible. In this manner data 28 can be arranged on information surface 25. The dark-coloured layer 26 and light-coloured layer 27 can of course be replaced by any other two contrasting colours so as to achieve an effective result. The advantage compared to other materials which can be coloured by electromagnetic radiation is that a much greater flexibility results for the colours and colour combinations of information surface 25 and the data 28 arranged hereon.

FIG. 2 shows a schematic view of a device 40 for arranging information on containers according to the invention. Device 40 comprises a hopper 41 in which a plurality of tissue containers 42 according to the invention is stacked. Using a conveyor 43 the tissue containers 42 are supplied to a marking device 44 which is provided with a laser 45 and a detection member 46 with which the position of a supplied tissue container 42 relative to laser 45 is determined. Laser 44 arranges data 47 in the form of letters, numbers and/or a bar code on tissue container 42. Arranging data using a laser 45 is quicker than a similar process using a plotter or printer, and a higher graphic resolution is also relatively easy to achieve. Marking device 44 is also provided with input means 48, such as a keyboard or a bar code reader, for inputting data 47, a storage medium 49 in which data 47 can be held, for instance in the form of a database, and a display 50 for displaying data 47. Device 40 makes it possible to arrange data 47 on containers according to the invention in relatively rapid manner.

It will be apparent that many variations and applications are still possible within the scope of the invention for a skilled person in the field.

1. Container for containing tissue, comprising
at least one receiving space for tissue, and
at least one information surface for arranging data,
characterized in that
at least the information surface is manufactured from a material which can be colored by electromagnetic radiation.

2. Container as claimed in claim 1, characterized in that the material which can be colored by electromagnetic radiation is formed substantially from a plastic material.

3. Container as claimed in claim 2, characterized in that the whole container is formed substantially from the plastic material.

4. Container as claimed in claim 2, characterized in that the plastic material comprises at least one of the following plastics: acetal copolymer, acrylonitrile butadiene styrene, nylon, polyacetal, polycarbonate, polyester, polypropylene, polyurethane, polystyrene, polyphenylene sulphide, polyethylene terephthalate, polybutylene terephthalate and polyoxymethylene.

5. Container as claimed in claim 2, characterized in that the plastic material substantially comprises polyacetal.

6. Container as claimed claim 1, characterized in that the material which can be colored by electromagnetic radiation comprises a radiation-absorbing pigment.

7. Container as claimed in claim 6, characterized in that the radiation-absorbing pigment comprises at least one of the following components: mica, pearl pigment, kaolin, aluminium metal, aluminium silicate, antimony trioxide, iron oxide, tin oxide, titanium oxide and aluminium hydroxide.

8. Container as claimed in claim 1, characterized in that the material which can be colored by radiation is formed by a laminate comprising at least two layers having mutually contrasting colors.

9. Container as claimed in claim 1, characterized in that the information surface can be coupled releasably to the container.

10. Container as claimed in claim 1, characterized in that the container is provided with a reference.

11. Container as claimed in claim 1, characterized in that the information surface is provided with data arranged by selective coloring of the material which can be colored by radiation.

12. Device for arranging information on a container for tissue as claimed in claim 1, comprising;
marking means for arranging data on an information surface of the container, and
positioning means for relative positioning of the information surface and the marking means,
characterized in that the marking means comprise an electromagnetic radiation source.

13. Device as claimed in claim 12, characterized in that the electromagnetic radiation source is a laser.

14. Device as claimed in claim 12, characterized in that the positioning means comprise detection means for determining the position of the information surface of the container.

15. Device as claimed in claim 12, characterized in that the marking means are connected to a database win data

16. Method for arranging data on a container for tissue as claimed in claim 1, comprising the following processing steps of:
positioning an information surface of the container and an electromagnetic radiation source relative to each other, and
arranging data on the information surface by selective coloring of at least a part of the information surface by means of electromagnetic radiation.

17. Method as claimed in claim 16, characterized in that a laser is used as electromagnetic radiation source.

18. Method as claimed in claim 16, characterized in that the data are arranged using a device comprising:
marking means for arranging data on an information surface of the container, the marking means comprising an electromagnetic radiation source, and
positioning means for relative positioning of the information surface and the marking means.
